(19) United States
(12) Patent Application Publication
(10) Pub. No.: US 2015/0290000 A1
(43) Pub. Date: Oct. 15, 2015

(54) MAGNETICALLY EXPANDABLE MEDICAL DEVICE

(71) Applicant: Cook Medical Technologies, LLC, Bloomington, IN (US)

(72) Inventors: Palle Munk Hansen, Bjaerverskov (DK); Torben Peter Andersen, Taastrup (DK)

(21) Appl. No.: 14/684,681

(22) Filed: Apr. 13, 2015

(30) Foreign Application Priority Data
Apr. 14, 2014 (GB) 1406658.3

(51) Int. Cl. A61F 2/82; A61F 2/04

(52) U.S. Cl. CPC A61F 2/82; A61F 2/04; A61F 2210/009; A61F 2210/0004

(57) ABSTRACT

An implantable medical device includes a body member, which may be a graft tube, is provided with a plurality of magnetic elements disposed in opposing sets and arranged with opposing magnetic polarities. The magnetic elements create repulsive forces which open the implantable medical device, providing an alternative to a conventional implantable medical device which is kept open by mechanical forces.

### RELATED APPLICATIONS

This application claims the benefit of priority under 35 U.S.C. § 119(a) to Great Britain Patent Application No. 1406658.3, filed on Apr. 14, 2014, which is incorporated by reference here in its entirety.

### TECHNICAL FIELD

The present invention relates to a magnetically expandable medical device, preferably an implantable medical device, and in the preferred embodiment to a magnetically expandable graft. The teachings herein can also be used in a soft catheter or sleeve, for instance for an introducer assembly.

### BACKGROUND ART

Implantable medical devices are known in many forms and for treating many medical conditions. Examples include stents, grafts, filters, occluders, valve replacement prostheses and so on. Such devices are generally introduced into the patient endoluminally through a remote percutaneous entry point. In order to achieve this, the medical device is loaded onto a carrier at a distal end of an introducer assembly with the device being held in a radially compressed configuration. The introducer assembly is fed into the patient's vasculature from the percutaneous entry point until its distal end is located at the treatment site. Once so positioned, the medical device is released from the carrier and expanded until the device engages the vessel wall to be held thereby. The device can be of a type which expands automatically, achieved by use of spring material, shape memory material and so on. Other types of device are plastically deformable and expanded by a separate mechanism, for instance by inflation of a delivery balloon on which the device is held in crimped form.

It is important that in use the medical device applies a constant force against the walls of the vessel in which it is located. This ensures good patency to the vessel wall, that is a good seal between the device and the wall tissue, in order to stop leakage around the device. The application of constant force also ensures that the device does not migrate or rotate out of alignment over time.

The force produced by the above-mentioned medical devices is a mechanical force, be it by a spring force of the components of the device or by relative mechanical stiffness in the case of plastically deformable devices. This requires the devices to have a certain structural strength and as a result a certain volume of material, resulting in increased device profile and reduced compressibility for delivery purposes. In addition, such devices have a relatively high volume of foreign material which is implanted and left in the patient's body.

Furthermore, the structure of such devices can impart unnatural forces on the vessel wall, the most common being a vessel straightening force acting against the natural curvature of the vessel or a significant pressing force against the sides of the vessel. Such forces can lead to restenosis of the vessel.

Some examples of implantable medical devices are disclosed in U.S. 2011/0257724, U.S. 2006/0212113, U.S. Pat. No. 8,449,604 and U.S. Pat. No. 7,722,668.

### SUMMARY OF THE INVENTION

The present invention seeks to provide an improved implantable medical device and an introducer assembly therefor. The device may be, but is not limited to, a vascular graft.

According to an aspect of the present invention, there is provided a medical device including an expandable body member having at least first and second sides with opposing internal surfaces, at least first and second magnetic elements each located at a respective one of said opposing internal surfaces as to be disposed in opposing relation to one another, wherein each magnetic element has a north pole and a south pole, the magnetic elements being disposed so as to have similar poles facing one another thereby to generate a repulsive force between one another.

The device is preferably an implantable medical device, such as a prosthesis.

A medical device of such a structure does not need to rely upon the generation of a mechanical force as with conventional medical devices, but instead makes use of constant magnetic repulsion to keep the body member of the device in its expanded state against the vessel wall. As a result of the use of such magnetic forces it is not necessary to generate large opening forces to hold the device in place. Moreover, the device can in practice be much more flexible and able to configure to the shape of the vessel or other organ, as well as accommodating changes in the vessel during normal body function and over time. This enhanced flexibility also makes the device suitable for the cerebral vessels.

The device may be any expandable prosthesis. It may be a graft, stent, filter, occluder, valve replacement prosthesis, for example. The teachings herein could also be used for other medical devices, such as catheters and the like, enabling the provision of a soft catheter for delicate vessels for instance.

In an embodiment, the body member has a tubular or conical shape and may be formed of woven, knitted, braided or sheet material.

The device may be or include a graft, the graft forming the body member.

The magnetic elements are preferably disposed such that their south poles face in the same direction as the internal surfaces of the body member, although could be oriented in the opposite direction.

In one embodiment, the magnetic elements are disks or rods, which may be generally circular, although could have other shapes such as oval, square or rectangular.

There may be provided at least first and second lines of said magnetic elements along the body portion of the device. Preferably, there may be provided at least two pairs of lines of magnetic elements, the lines in each pair being disposed on opposing internal surfaces of the body member. For example, there may be provided four, six or eight lines of magnetic elements, arranged in opposing pairs.

In one embodiment, the lines of magnetic elements extend parallel to an axial or longitudinal dimension of the body member, although in another embodiment the lines of magnetic elements extend at an angle to the axial dimension of the body member. They may, for example, extend helically or in a zigzag manner along the body member. In some embodiments, the magnetic elements may extend circumferentially around the body member, as a simple annular shape or in any other shape including undulating and zigzag.

In another embodiment, the magnetic elements are strips of magnetic material, which may extend parallel to the axial or longitudinal dimension of the body member or at an angle thereto. The strips of magnetic material may have the same shapes as characteristics as the lines of magnetic elements. Furthermore, the strips may be discontinuous along the length of the body member.

The magnetic elements may be painted on or attached to the body member of the device.

In a preferred embodiment, the magnetic elements are formed of paramagnetic material, which can be magnetised once the elements have been fitted to the body member of the device.

Advantageously, the magnetic elements are formed of biodegradable material, preferably of a material which will degrade at a rate slower than a rate of ingrowth of vessel tissue.

Other features of the apparatus and method disclosed herein will become apparent from the following specific description of preferred embodiments.

### BRIEF DESCRIPTION OF THE DRAWINGS

Embodiments of the present invention are described below, by way of example only, with reference to the accompanying drawings, in which:

FIG. 1 is a schematic view in perspective of an embodiment of graft according to the invention;

FIG. 2 is a transverse cross-sectional view of the graft of FIG. 1;

FIG. 3 is a schematic view in perspective of another embodiment of graft according to the invention;

FIG. 4 is a schematic view of the graft of FIG. 1 disposed in a vessel, the vessel being shown in cross-section;

FIG. 5 is a schematic view of the graft of FIG. 1 disposed in a curved vessel, the vessel being shown in cross-section; and

FIG. 6 is a schematic view of one of the magnetic elements of the device of FIG. 1.

### DESCRIPTION OF THE PREFERRED EMBODIMENTS

The embodiments described below are directed to a graft of tubular and substantially cylindrical form. The teachings herein are particularly suited to vascular grafts, although it is to be understood that the teachings herein are not limited to a graft of this structure and are equally applicable to grafts having a non-uniform shape and/or size along its length, such as a tapering graft, a graft with a central waist and so on. Equally, the teachings herein are also applicable to other forms of medical device, including devices provided with graft elements or other coverings and also medical devices having no graft element, such as stents and so on.

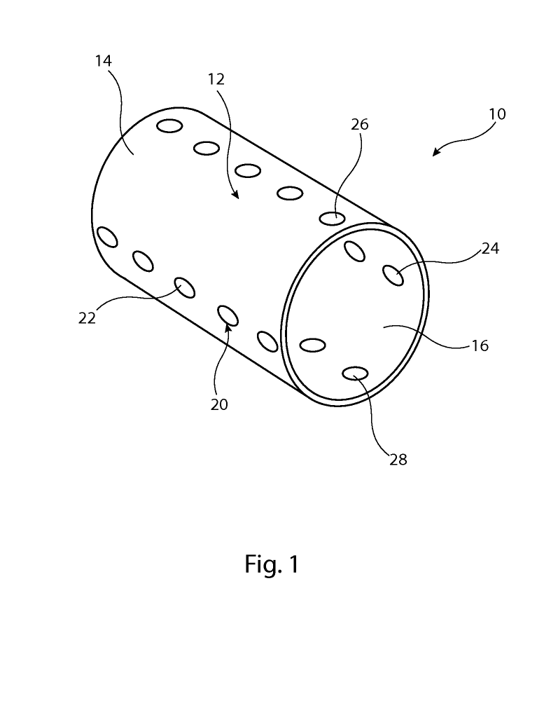

Referring first to FIG. 1, the graft 10 shown in schematic form includes a body member 12 of generally tubular form and made of, in this example, a conventional graft material, typically woven or knitted. The graft material could be of ultrahigh molecular weight polyethylene, such as Dyneema™, Expanded polytetrafluoroethylene (EPTFE) or any other suitable or known material. The tubular graft element 12 includes an outer abluminal surface 14 and an inner luminal surface 16. Attached to graft element 12 is a plurality of magnetic elements 20. The elements 20 are, in this example, arranged in four arrays or lines 22, 24, 26 and 28 which extend along the length of the tubular graft element 12 and in this embodiment substantially parallel to the longitudinal axis of the graft element 12. The arrays or lines 22-28 of magnetic elements 20 are arranged in opposing pairs. More specifically, the arrays or lines 22 and 24 are disposed on opposite sides of the internal surface 16 of the graft element 12 and the pair of arrays or lines 26 or 28 are likewise disposed opposite one another. In this example, the four arrays or lines 22, 28 are equally spaced circumferentially around the graft tube 12 so in this example separated from one another by 90° around the graft tube. As is described in further detail below, the magnetic elements 20 each have north and south poles and are disposed on the graft tube such that they have the same pole facing inwardly, towards the central axis of the graft tube. In one embodiment, the south pole of the magnetic elements 20 faces towards the radial centre of the graft tube 12. Thus, all of the magnetic elements 20 have the opposing pole, in this example, the north pole, facing outwardly of the graft tube. Of course, the polarities of all the magnets could be reversed relative to those depicted.

The magnetic elements could in their simplest form be formed of a magnetic material printed or otherwise applied to the material of the graft tube 12 or can be magnetic disks or other three-dimensional structures mechanically affixed to the graft tube 12, for instance by bonding, riveting or the like.

There may be provided at the ends of the graft tube 12 first and second stents for keeping the ends of the graft fully open, thereby ensuring patency with the vessel wall. Suitable stents include stents having a zigzag form for radial compressibility. Each stent could be sewn to a respective end of the graft tube 12 and disposed either inside or outside the graft tube 12. Such stents can be useful in embodiments in which only a few magnetic elements 20 are disposed circumferentially around the graft.

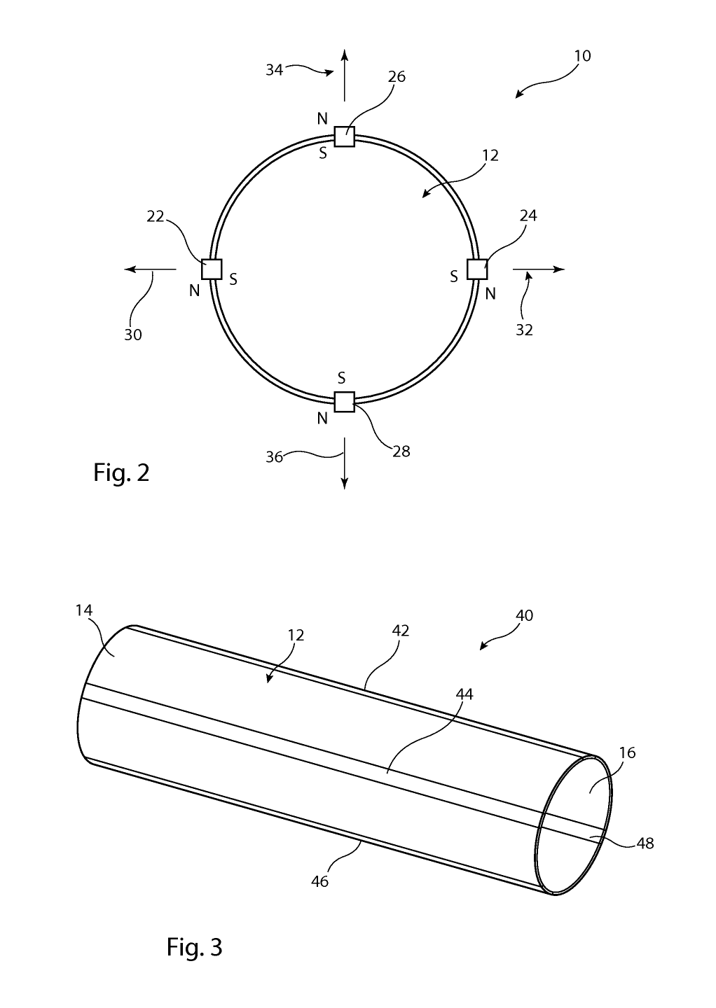

Referring now to FIG. 2, this shows a cross-sectional view of the graft of FIG. 1, in schematic form. FIG. 2 shows arrows 30-36 which depict the magnetic force generated at each of the magnetic elements 22-28 as the result of the opposing magnetic relation with the corresponding magnetic element on the opposing side of the graft tube 12. More specifically, in the described embodiment, the facing south poles of the magnetic elements 22, 24 will produce mutually repulsive magnetic forces in the directions of arrows 30 and 32. Likewise, the opposing magnetic elements 26 and 28 will, by virtue of having their south poles facing one another, also generate mutually repulsive magnetic forces 34 and 36 in opposing directions. These repulsive magnetic forces 30-36 will cause the graft element to be biased in the open configuration, as will be apparent from the schematic diagram of FIG. 2.

It will be appreciated that it is preferred that each magnetic element 20 has a directly facing counterpart on the opposing side of the graft tube 12, that is opposing in the radial direction as well in the longitudinal direction along the length of the graft element 12. It is not excluded, however, that the magnetic elements 20 could be slightly offset relative to their opposing counterpart, either in the circumferential direction or in the longitudinal direction or both. However, the offset will produce a lower repulsive force.

FIGS. 1 and 2 depicted an embodiment having two sets of magnetic elements 22, 24 and 26, 28. It is envisaged that some embodiments may have more than two pairs of sets of magnetic elements 20, for instance 4, 6, 8 or more pairs, which again are preferably evenly spaced circumferentially around the tubular graft element 12. Each opposing set of magnetic elements would produce repulsive magnetic forces as with the embodiment of FIG. 2, with the addition of further magnetic repulsion between adjacent magnetic elements 20, enhancing the opening of the medical device 10. It is to be understood, though, that in most embodiments it is not necessary to have many magnetic elements 20 on the graft tube 12 as blood flow will tend to keep the graft tube open, the magnetic elements being provided to ensure there is no collapse of the graft tube.

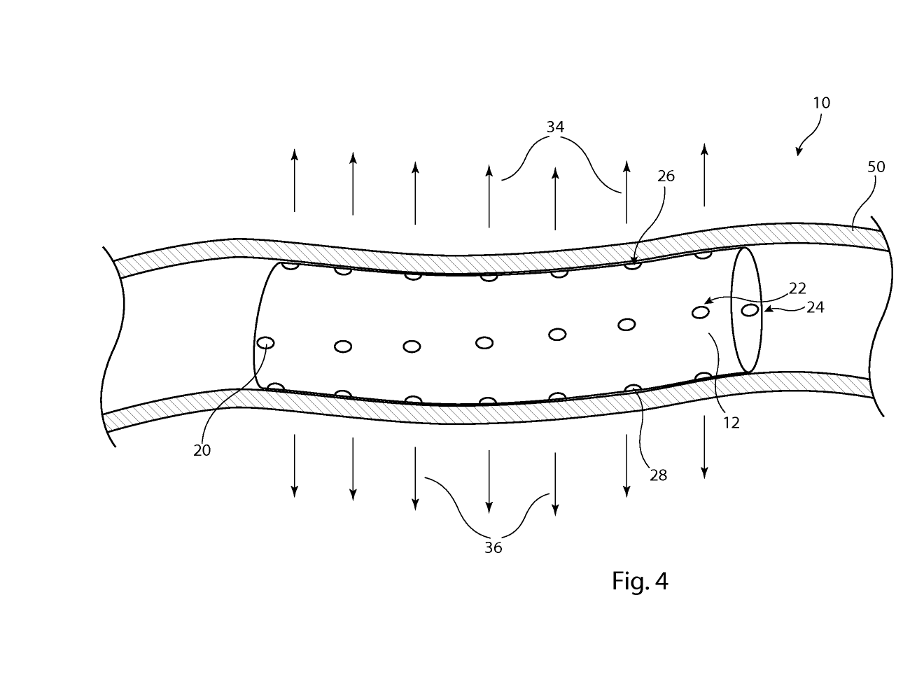

Referring now to FIG. 3, this shows another embodiment of implantable medical device 40, which again has a tubular graft element 12 similar to that of the embodiment of FIGS. 1 and 2 and having similar characteristics. The disk-shaped magnetic elements 22 of the embodiment of FIGS. 1 and 2 are replaced by strips of magnetic material 42-48, which extend along the length of the graft tube 12, which forms the body portion of the medical device 40. The strips of magnetic material 42-48 extend, in this embodiment, substantially parallel to the longitudinal axis of the tubular graft element 12 and are arranged in opposing pairs, that is with the strips 42, 46 being on opposing sides of the graft tube 12, the strips 44 and 48 extending in like manner. The strips 42-48 have north and south polarities and are arranged such that the same polarity faces internally into the luminal side 16 of the graft tube 12 and the opposite polarity faces the abluminal side 16. In one example, the south pole of each magnetic strip 42-48 extends towards the interior of the graft tube 12 and the north pole faces to the exterior. This arrangement of strips of magnetic material 42-48 provides a very similar functionality to the embodiments of FIGS. 1 and 2, with a smaller number of magnetic elements being necessary. Again, these strips of magnetic material 42-48 could be provided by printing magnetic material onto the graft tube or by affixing three-dimensional strips to the graft tube.

In the embodiment of FIG. 3, the strips 42-48 are continuous along the whole length of the graft element 12, although it is not excluded that these could be discontinuous, that is segmented with or without a gap between adjacent segments of each strip 42-48. Segmenting the strips of magnetic material 42-48 can enhance the longitudinal flexibility of the medical device 40, as will be apparent from the teachings below.

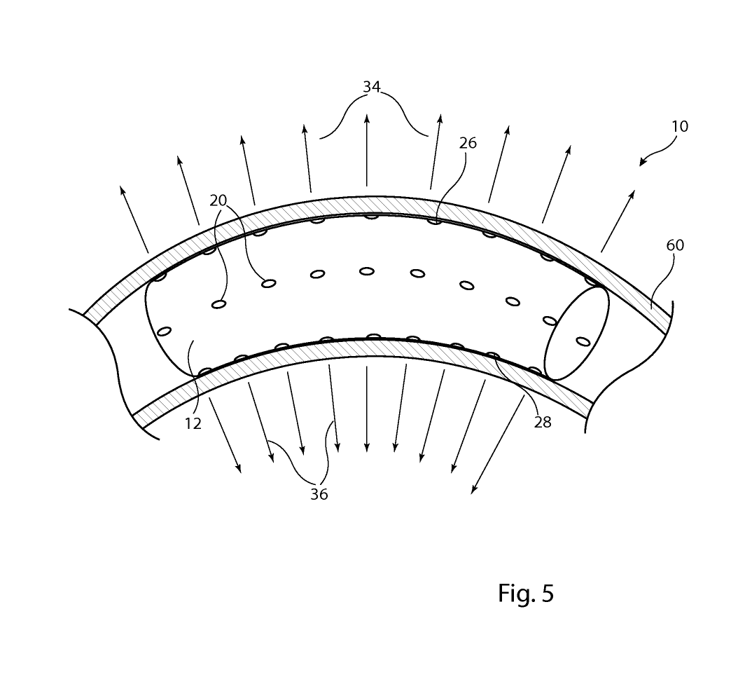

Referring now to FIG. 4, this shows the implantable medical device 10 of FIGS. 1 and 2 disposed within a vessel 50 of a patient. The vessel 50 is shown in cross-section, whereas the implantable medical device 10 is shown in its complete form. FIG. 4 also shows the direction of the magnetic repulsive forces 34 and 36 generated by the opposing sets 26, 28 of magnetic elements 20 and the skilled person will appreciate that the equivalent opposing magnetic forces will be generated also at the sets 22 and 24 of magnetic elements and any other opposing sets of magnetic elements. The opposing magnetic forces 34, 36 cause the graft element 12 to be pressed against the vessel wall 50 and thereby ensure that the tubular graft element 12 remains open within the vessel. Furthermore, the repulsive magnetic forces provide a constant opening force to the medical device 10 irrespective of other factors such as vessel movement and the like. As the opening force is magnetic, it is not necessary for the magnetic elements 20 to be particularly large and the graft tube 12 (or other structure of another form of medical device) similarly need not be thick or otherwise voluminous. The medical device 10, therefore, can be very thin, as a result presenting virtually no or otherwise minimal disruption to the flow of fluid within the vessel 50. This can also minimise the amount of foreign material in the body. Moreover, these characteristics also make the device suitable for smaller vessels.

Referring now to FIG. 5, this shows the implantable medical device 10 deployed within a curved vessel 60. The medical device 10 is the same as that of FIG. 4 and the embodiments of FIGS. 1 and 2, although it is not excluded that the device 10 could be shaped to correspond with the shape of the vessel 60.

As will be apparent from the force arrows 34, 36 in FIG. 5, the opposing magnetic elements will generate the same repulsive magnetic forces but these will be directed in the orthogonal direction relative to the axis vessel 60, thereby ensuring that the graft tubing 12 remains consistently pressed against the vessel wall along the curve, thereby keeping the graft tubing 12 open and in position. These opposing repulsive magnetic forces will therefore be aligned with the curvature of the vessel 60, as depicted in FIG. 5. With the embodiment of FIGS. 1 and 2 as well as with any other embodiment of medical device having segmented or otherwise separate magnetic elements along the length of the graft tubing 12, the structure of device will not exert any straightening force on the vessel 60 of the type which is experienced with conventional implantable medical devices. This makes the medical device disclosed herein suitable for implantation in delicate vessels of a patient, including the cerebral vessels.

In the embodiments of medical device disclosed herein, the magnetic elements could be made from permanent magnets and equally could be made from a paramagnetic material and magnetised after fixation to the body portion of the medical device, for example by means of an electromagnet or a permanent magnetic field. The elements would be magnetised to ensure that the relative polarisation direction of each spot or magnetic strip is identical.

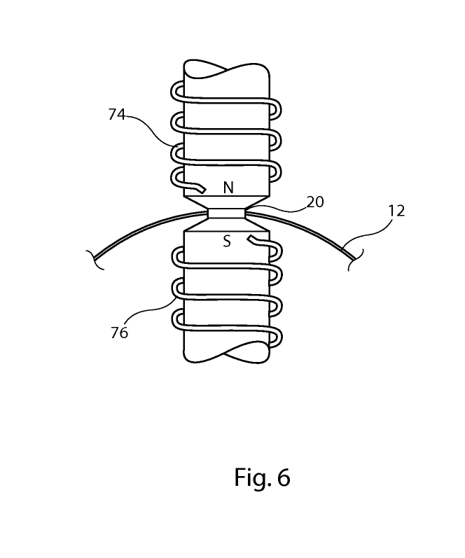

Referring now to FIG. 6, this shows an enlarged form, one of the magnetic elements 20 of the embodiment of FIGS. 1, 2, 4 and 5. In this example, the magnetic element 20 is in the form of a disk or rivet which extends through the body member 12. In this example, the magnetic element 20 is made of a paramagnetic material and magnetisation thereof is achieved by providing electromagnets 72, 74 either side of each magnetic element. The electromagnet 76 can usefully be in the form of a mandrel which is fed into the lumen of the graft. The electromagnet 74 could be a series of electromagnetic elements or a sleeve. The electromagnets 74 and 76 are energised to cause the paramagnetic material of the element 20 to be polarised, in this example to have its south pole on the interior surface of the body member 12 and its north pole extending outwardly of the body portion 12 and therefore of the device 10.

The method of magnetisation of a paramagnetic element 20 may be by means of a permanent magnet or electromagnetically.

In the embodiments described above, there are provided arrays or lines of magnetic elements or strips of magnetic element which extend parallel to the longitudinal axis of the body portion of the medical device 10. In other embodiments, the magnetic elements may extend at an angle to the longitudinal axis and could, for example, extend helically around the body portion or even in zigzag or other curved shape. The exact arrangement and/or shape of the magnetic elements can be varied to give the device 10 different opening characteristics. Equally, some parts of the body portion of the device 10 can be free of magnetic elements in some embodiments.

It is also envisaged that the magnetic elements could extend circumferentially around the body portion, in one or more annular bands. Each strip could be simply annular or undulating or of zigzag form.

It is envisaged that the medical device 10 could be deployed in a patient's vessel over an inflatable delivery balloon. Such a balloon can ensure that the body member 12 of the device is expanded properly against the vessel wall, with the magnetic elements 20 then ensuring that the device 10 expands as desired.

The magnetic elements could be made of: NdFeB (neodymium), FeCrCo, SMCo or PtCo.

It is envisaged that the magnetic elements could have a thickness of 0.10 millimetres or more.

It is preferred that the magnetic elements are formed from a biodegradable or bioabsorbable material, likewise with the other elements of the device.

It is not necessary for the medical device to have a graft element. In some embodiments the magnetic elements could be fitted, for example, to a stent or other open structure, for example allowing the stent to have a smaller and weaker mechanical structure than conventional stents.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

The disclosure in the abstract accompanying this application is incorporated herein by reference.

1. A medical device including an expandable body member including at least first and second sides with opposing internal surfaces, at least first and second magnetic elements each located at a respective one of said opposing internal surfaces as to be disposed in opposing relation to one another, wherein each magnetic element has a north pole and a south pole, the magnetic elements being disposed so as to have similar poles facing one another thereby to generate a repulsive force between one another.

2. A medical device according to claim 1, wherein the device is an implantable medical device.

3. A medical device according to claim 1, wherein the body member has a cylindrical tubular or conical shape.

4. A device according to claim 1, wherein the body member is formed of a first material chosen from the group of a knitted, a woven, a braided, and a sheet material.

5. A device according to claim 1, wherein the device includes a graft, the graft including said body member.

6. A device according to claim 1, including a stent at either end of the body member.

7. A device according to claim 1, wherein the magnetic elements are disks or rivets.

8. A device according to claim 7, including at least first and second lines of said magnetic elements along the body portion of the device.

9. A device according to claim 8, including at least two pairs of lines of magnetic elements, the lines in each pair being disposed on opposing internal surfaces of the body member.

10. A device according to claim 8, wherein the body member of the device has an axial dimension and said first and second lines of magnetic elements extend parallel to the axial dimension of the body member.

11. A device according to claim 8, wherein the body member of the device has an axial dimension and said first and second lines of magnetic elements extend at an angle to the axial dimension of the body member.

12. A device according to claim 11, wherein the first and second lines of magnetic elements extend helically around the body member.

13. A device according to claim 11, wherein the first and second lines of magnetic elements extend in a zigzag manner along the body member.

14. A device according to claim 1, wherein the magnetic elements are strips of magnetic material.

15. A device according to claim 14, wherein the body member of the device has an axial dimension and the strips of magnetic material extend parallel to the axial dimension of the body member.

16. A device according to claim 14, wherein the body member of the device has an axial dimension and the strips of magnetic material extend at an angle to the axial dimension of the body member.

17. A device according to claim 16, wherein the strips of magnetic material extend helically around the body member.

18. A device according to claim 16, wherein the strips of magnetic material extend in a zigzag manner along the body member.

19. A device according to claim 14, including at least two pairs of strips of magnetic material, the strips in each pair being disposed on opposing internal surfaces of the body member.

20. A device according to claim 14, wherein the strips of magnetic material are discontinuous along the body member.

21. A device according to claim 1, wherein the magnetic elements are painted on, printed on or attached to the body member of the device.

22. A device according to claim 1, wherein the magnetic elements are formed of paramagnetic material.

23. A device according to claim 1, wherein the magnetic elements are formed of biodegradable or bioabsorbable material.
